(19) United States
(12) Patent Application Publication
(10) Pub. No.: US 2024/0016870 A1
(43) Pub. Date: Jan. 18, 2024

(54) PROCESS FOR PREPARING CANNABIS SUGAR WAX CONCENTRATE AND RELATED COMPOSITIONS AND METHODS

(71) Applicant: MEDPHARM IOWA, LLC, Des Moines, IA (US)

(72) Inventors: Zach Joseph BAKER, West Des Moines, IA (US); Jack McCaslin CARRAHER, Wilmington, NC (US)

(73) Assignee: MEDPHARM IOWA, LLC, Des Moines, IA (US)

(21) Appl. No.: 18/221,987

(22) Filed: Jul. 14, 2023

Related U.S. Application Data
(60) Provisional application No. 63/389,704, filed on Jul. 15, 2022.

(51) Int. Cl. A61K 36/185; A61K 31/352

(52) U.S. Cl. CPC A61K 36/185; A61K 31/352; A61K 2236/17; A61K 2236/333; A61K 2236/35; A61K 2236/37

(57) ABSTRACT

The present invention relates to the manufacture of a Cannabis concentrate from dried, destemmed, and optionally ground Cannabis obtained via a closed-loop extraction using butane or propane. Another aspect of the present invention relates to a process of exposing dried, destemmed, and optionally ground Cannabis obtained via sequential CO₂ and closed-loop extraction using butane or propane. Another aspect of the present invention relates to exposing Cannabis biomass to a polar solvent, filtering, then evaporating the solvent to form an extract. The solvents are then removed, for instance using heat and vacuum. Another aspect of the invention relates to compositions, such as Cannabis concentrates, that can be obtained through the processes described.

### CROSS-REFERENCE TO RELATED APPLICATIONS

The present application claims the benefit of priority to U.S. Provisional Patent Application No. 63/389,704, entitled “Process for Preparing Cannabis Sugar Wax Concentrate and Related Composition and Methods,” filed Jul. 15, 2022, the entire disclosure of which is incorporated herein by reference in its entirety.

### BACKGROUND OF THE INVENTION

There is a long-felt need in the Cannabis industry for an effective means of (1) providing a Cannabis concentrate product that can be dispensed with a semi-automated filler; (2) providing a batch product that is consistent from jar to jar due to the ability to homogenize the mixture prior to dispensing; (3) providing a product that can be packaged in hours instead of days; and (4) high batch to batch consistency.

### BRIEF DESCRIPTION OF THE INVENTION

This summary is provided to introduce a selection of concepts that are further described below in the Detailed Description. This summary is not intended to identify key or essential features of the claimed subject matter, nor is it intended to be used as an aid in limiting the scope of the claimed subject matter.

The present invention relates to the manufacture of a Cannabis concentrate from dried, destemmed, and optionally ground Cannabis obtained via a closed-loop extraction using hydrocarbons, such as propane or butane. Another aspect of the present invention relates to a process of exposing dried, destemmed, and optionally ground Cannabis obtained via sequential CO₂ and closed-loop extraction using hydrocarbons, such as propane or butane. Another aspect of the present invention relates to exposing Cannabis biomass to a polar solvent, filtering, then evaporating the solvent to form an extract. The solvents are then removed, for instance using heat and vacuum. Another aspect of the invention relates to compositions, such as Cannabis concentrates, that can be obtained through the process described.

According to at least one embodiment, the present invention relates to methods for production of a Cannabis concentrate (sugar wax) using Cannabis biomass (THCA crystals) obtained from a “PiggyBack extraction” of closed-loop extraction process. The THCA crystals from the PiggyBack process are dissolved in a polar solvent, and then mixed with in-house terpenes (IHT) (terpenes extracted from Cannabis or another plant/fruit/or other natural or synthetic source in the form of isolates or blends) to contain up to 8% w/w IHT. The mixture can be homogenized using a high shear mixer and then dispensed with a semi automated machine. The volatiles are then removed by heating the mixture at a temperature of at least 40° C. for a time period sufficient to purge the volatiles to a concentration below at least 5000 ppm, and according to certain embodiments below 2500 ppm, and in certain embodiments below 1000 ppm.

The resulting sugar wax product provides a Cannabis concentrate product that can be dispensed with a semi-automated vape filling robot, provides a batch product that is consistent from jar to jar in a fraction of the time it takes to hand weigh the product.

### DETAILED DESCRIPTION OF THE FIGURES

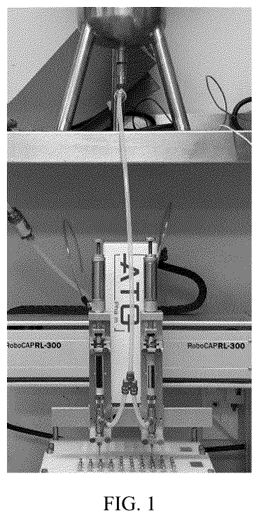

FIG. 1 is a photograph of a semi-automated filling machine and assembly for packaging of Sugar Wax.

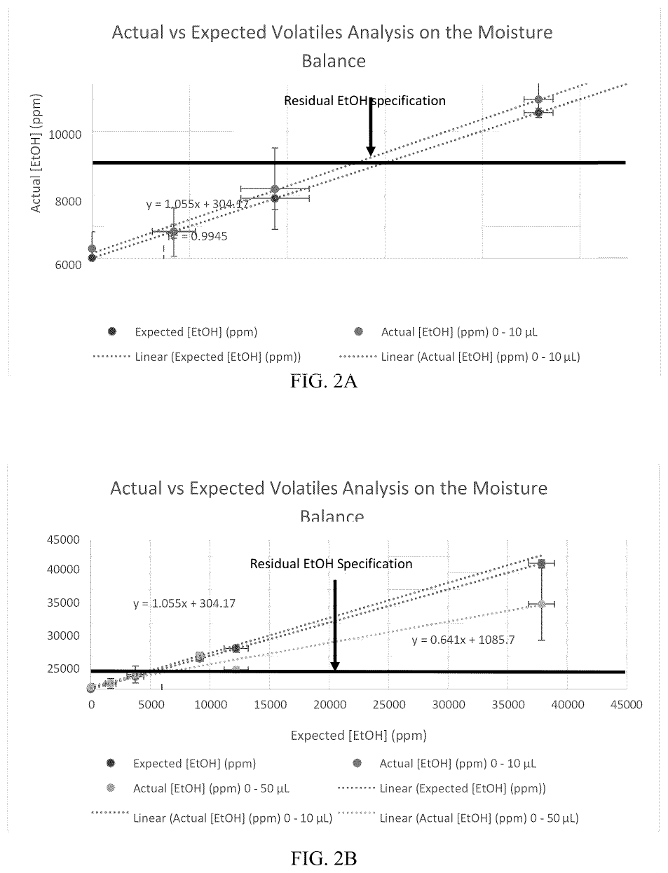

FIG. 2A is a graph illustrating a comparison of the actual versus expected amount of volatiles from spiking 0-10 μL of EtOH onto THCA crystals, as described in greater detail in Example 1.

FIG. 2B is a graph illustrating a comparison of the actual vs. expected amount of volatiles from spiking 0-10 μL of EtOH onto THCA crystals as described in greater detail in Example 1.

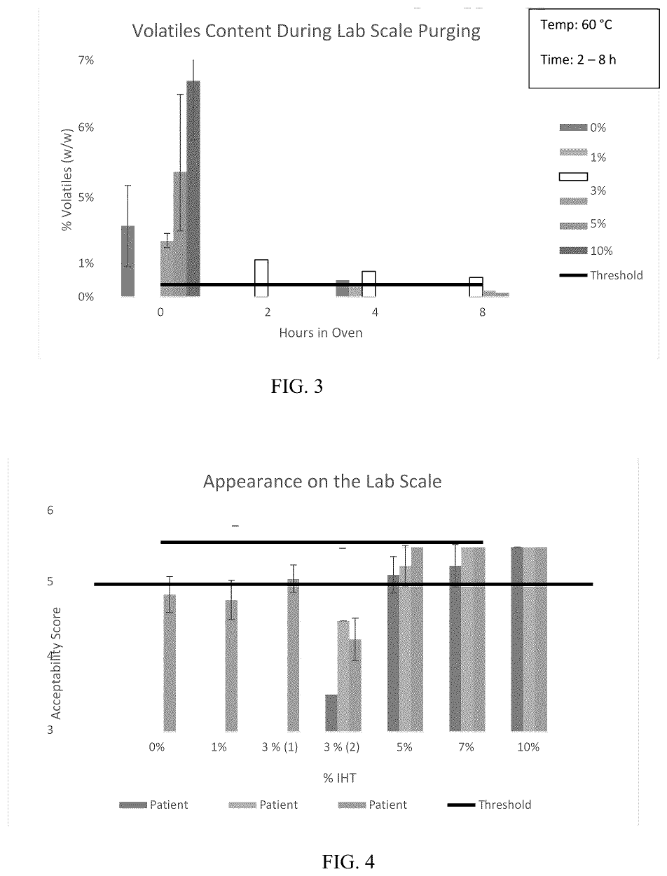

FIG. 3 is a graph illustrating the volatiles content during vacuum purging of lab scale formulations prepared with 0, 1, 3, 5, 7 and 10% (w/w) IHT.

FIG. 4 is a graph illustrating the acceptability of the appearance of each lab scale formulation by w/w % of IHT.

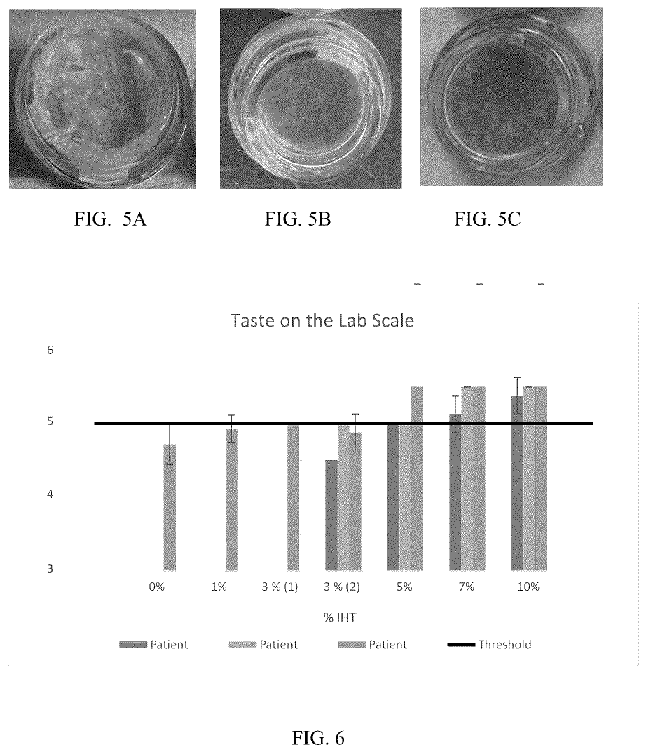

FIG. 5A is a photograph showing the appearance of lab scale formulations made with 3% IHT after vacuum purging for 8 h at 60° C. as set forth in Example 1.

FIG. 5B is a photograph showing the appearance of lab scale formulations made with 3% IHT after vacuum purging for 8 h at 60° C. as set forth in Example 1.

FIG. 5C is a photograph showing the appearance of lab scale formulations made with 10% IHT (right) after vacuum purging for 8 h at 60° C. as set forth in Example 1.

FIG. 6 is a graph illustrating the acceptability of the taste of each lab scale formulation by w/w % of IHT as set forth in Example 1.

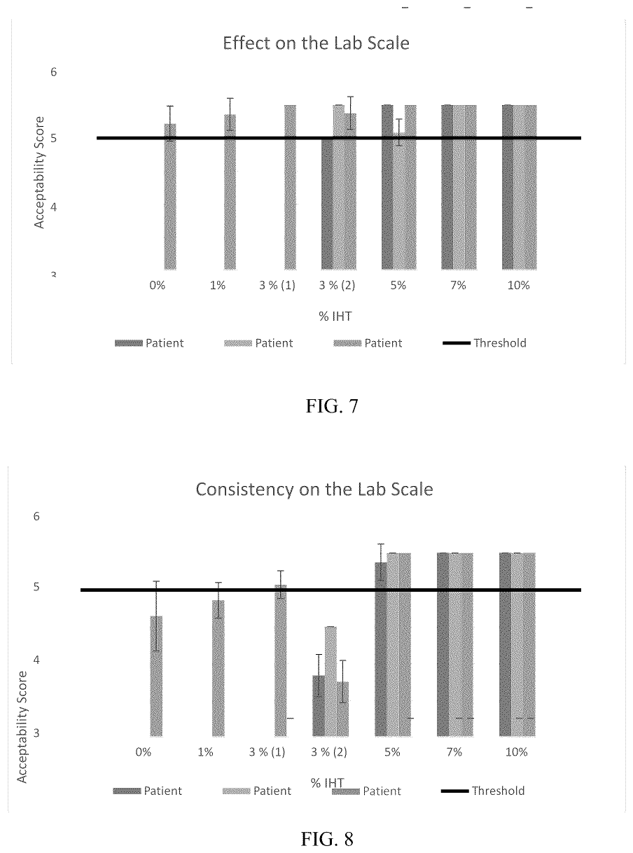

FIG. 7 is a graph illustrating the acceptability of the effect of each lab scale formulation by w/w % of IHT as set forth in Example 1.

FIG. 8 is a graph illustrating the acceptability of the consistency of each lab scale formulation by w/w % of IHT as set forth in Example 1.

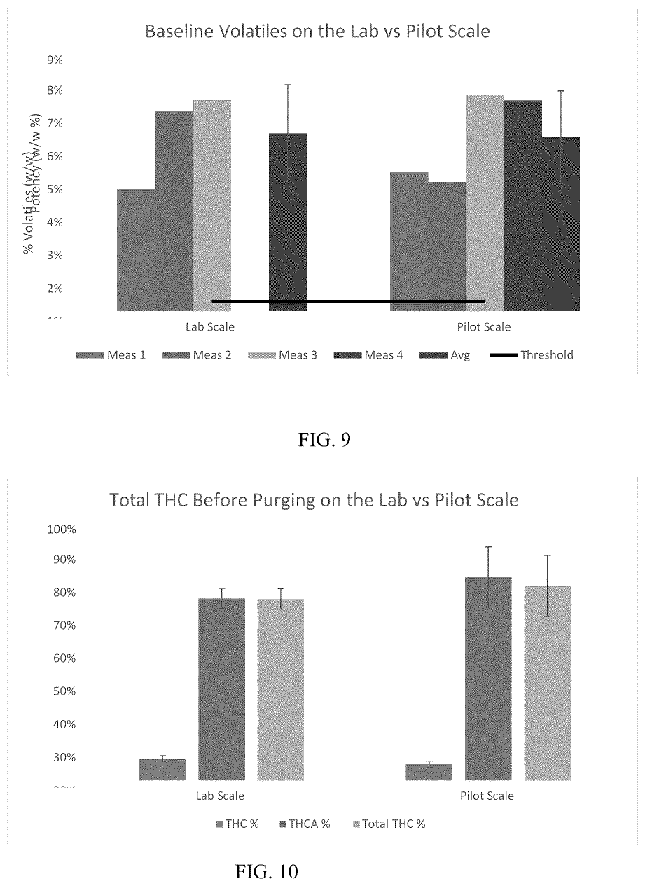

FIG. 9 is a graph illustrating the volatiles measurements for the lab scale vs pilot scale formulation before purging in the vacuum oven as set forth in Example 1.

FIG. 10 is a graph illustrating a comparison of the potency for THC %, THCA %, and total THC % between the lab scale and pilot scale formulations prior to purging in the vacuum oven, as set forth in Example 1.

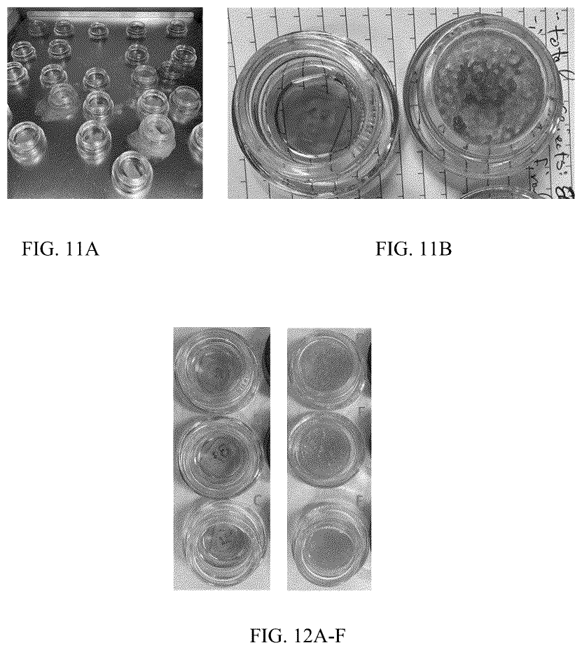

FIG. 11A is a photograph illustrating the appearance of the packaged units from the first pilot scale batch after 24 h in the vacuum oven as set forth in Example 1.

FIG. 11B is a photograph illustrating the appearance of the packaged units from the first pilot scale batch after 24 h in the vacuum oven as set forth in Example 1.

FIG. 12A is a photograph illustrating the appearance of the packaged units from the second pilot batch without a seed crystal after 9 h in the vacuum oven as set forth in Example 1.

FIG. 12B is a photograph illustrating the appearance of the packaged units from the second pilot batch without a seed crystal after 9 h in the vacuum oven as set forth in Example 1.

FIG. 12C is a photograph illustrating the appearance of the packaged units from the second pilot batch without a seed crystal after 9 h in the vacuum oven as set forth in Example 1.

FIG. 12D is a photograph illustrating the appearance of the packaged units from the second pilot batch with a seed crystal after 9 h in the vacuum oven as set forth in Example 1.

FIG. 12E is a photograph illustrating the appearance of the packaged units from the second pilot batch with a seed crystal after 9 h in the vacuum oven as set forth in Example 1.

FIG. 12F is a photograph illustrating the appearance of the packaged units from the second pilot batch with a seed crystal after 9 h in the vacuum oven as set forth in Example 1.

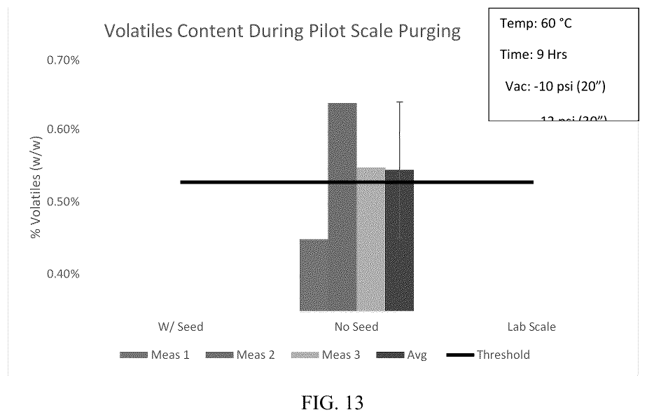

FIG. 13 is a graph illustrating a comparison of the % volatiles between jars with and without a seed crystal in the second pilot scale batch after 9 hours in the vacuum oven as set forth in Example 1. Also presented are the % volatiles from the lab scale experiment for comparison.

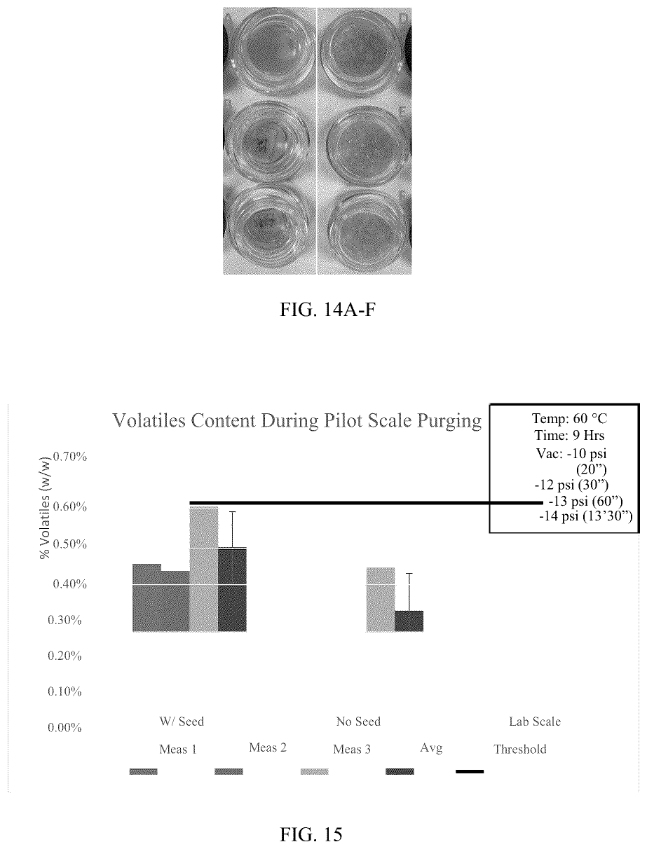

FIG. 14A is a photograph illustrating the appearance of the packaged units from the second pilot batch without a seed crystal after 15.5 hours in the vacuum oven as set forth in Example 1.

FIG. 14B is a photograph illustrating the appearance of the packaged units from the second pilot batch without a seed crystal after 15.5 hours in the vacuum oven as set forth in Example 1.

FIG. 14C is a photograph illustrating the appearance of the packaged units from the second pilot batch without a seed crystal after 15.5 hours in the vacuum oven as set forth in Example 1.

FIG. 14D is a photograph illustrating the appearance of the packaged units from the second pilot batch with a seed crystal after 15.5 hours in the vacuum oven as set forth in Example 1.

FIG. 14E is a photograph illustrating the appearance of the packaged units from the second pilot batch with a seed crystal after 15.5 hours in the vacuum oven as set forth in Example 1.

FIG. 14F is a photograph illustrating the appearance of the packaged units from the second pilot batch with a seed crystal after 15.5 hours in the vacuum oven as set forth in Example 1.

FIG. 15 is a graph illustrating a comparison of the % volatiles between jars with and without a seed crystal in the second pilot scale batch after 15.5 hours in the vacuum oven as set forth in Example 1.

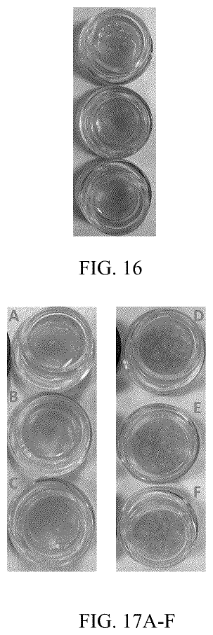

FIG. 16 are photographs illustrating the appearance of the packaged units from the second pilot batch without a seed crystal after 40 h in the vacuum oven as set forth in Example 1.

FIG. 17A is a photograph illustrating the appearance of the packaged units from the second pilot batch that were seeded after 40 h in the vacuum oven.

FIG. 17B is a photograph illustrating the appearance of the packaged units from the second pilot batch that were seeded after 40 h in the vacuum oven.

FIG. 17C is a photograph illustrating the appearance of the packaged units from the second pilot batch that were seeded after 40 h in the vacuum oven.

FIG. 17D is a photograph illustrating the appearance of the packaged units from the second pilot batch that were seeded immediately after packaging and vacuum purged for 15.5 h.

FIG. 17E is a photograph illustrating the appearance of the packaged units from the second pilot batch that were seeded immediately after packaging and vacuum purged for 15.5 h.

FIG. 17F is a photograph illustrating the appearance of the packaged units from the second pilot batch that were seeded immediately after packaging and vacuum purged for 15.5 h.

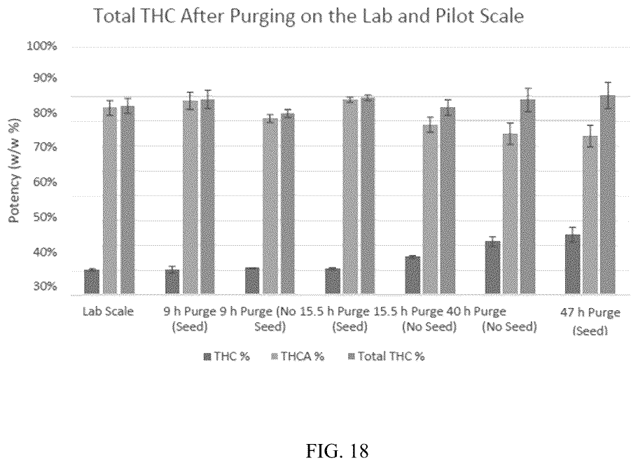

FIG. 18 is a graph illustrating a comparison of the potency for THC %, THCA %, and total THC % between the lab scale and pilot scale formulations after purging in the vacuum oven as set forth in Example 1.

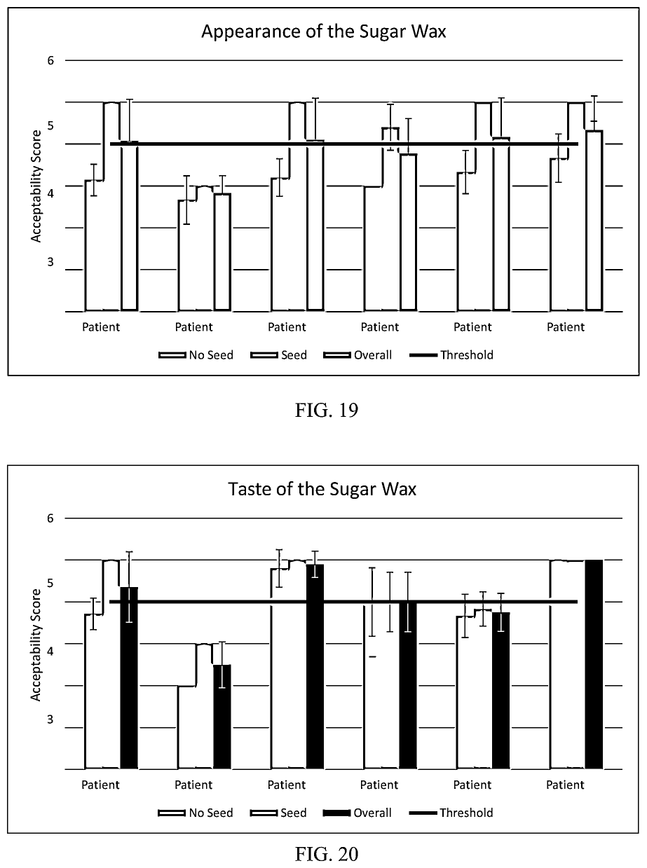

FIG. 19 is a graph illustrating the acceptability of the appearance of the pilot scale formulation by patient as set forth in Example 1.

FIG. 20 is a graph illustrating the acceptability of the taste of the pilot scale formulation by patient as set forth in Example 1.

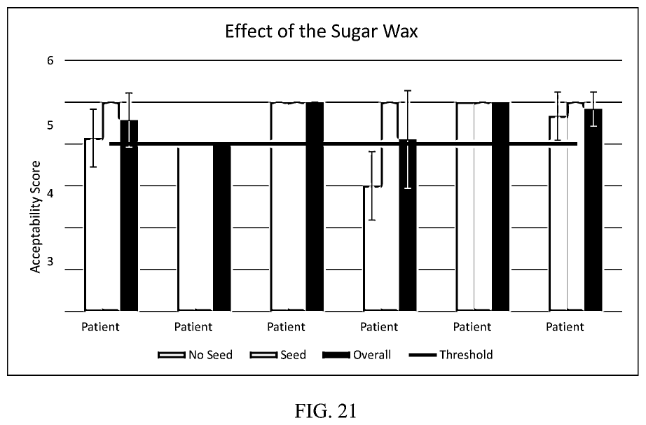

FIG. 21 is a graph illustrating the acceptability of the effect of the pilot scale formulation by patient as set forth in Example 1.

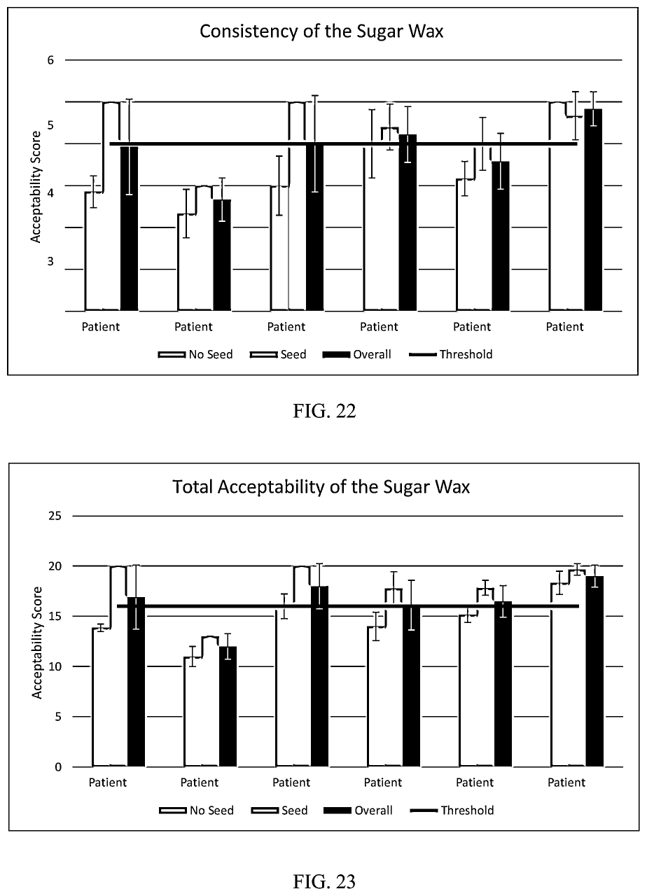

FIG. 22 is a graph illustrating the acceptability of the consistency of the pilot scale formulation by patient as set forth in Example 1.

FIG. 23 is a graph illustrating the total acceptability of the pilot scale formulation by patient as set forth in Example 1.

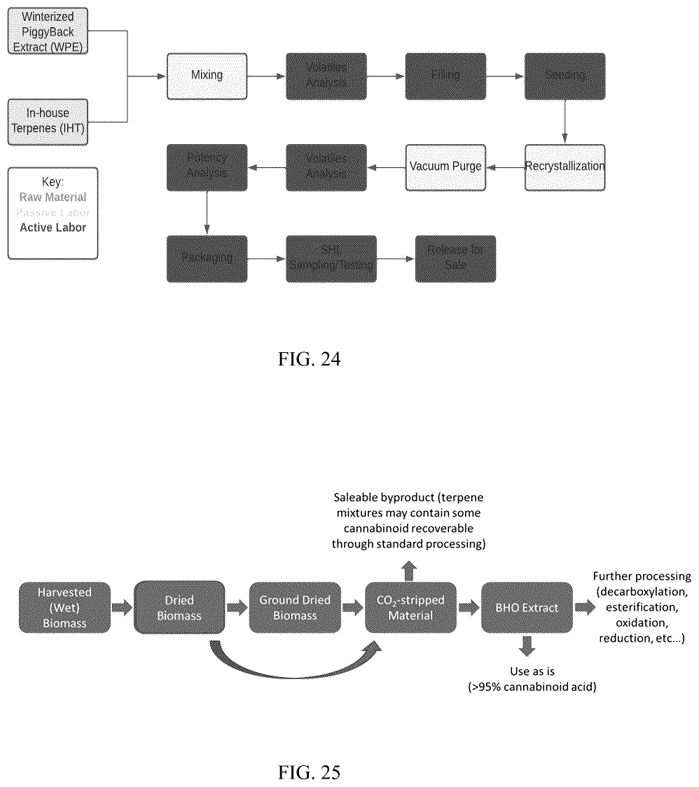

FIG. 24 is a flow chart illustrating the manufacturing steps of the sugar wax of the invention.

FIG. 25 is a process flow diagram illustrating a piggy-back extraction process.

### DETAILED DESCRIPTION OF THE INVENTION

The present invention describes a novel method for the production of Cannabis concentrate product (sugar wax) utilizing a raw material produced from the applicant's proprietary piggy-back extraction process described in U.S. Pat. No. 11,697,078 (U.S. Ser. No. 17/484,718) (“the '078 patent” and alternatively referred to herein as “PiggyBack” extraction process) filed Sep. 24, 2021, the disclosure of which is hereby specifically incorporated by reference in its entirety.

The “PiggyBack” extraction process, or closed-loop extraction process, described in the '078 patent requires harvesting of Cannabis plants, drying of biomass through either air-drying or by low temperature vacuum oven, destemming (optional, but recommended for efficiency), stripping terpenes and other cannabinoid or non-cannabinoid impurities with subcritical &/or supercritical CO₂, extraction of high purity cannabinoids/cannabinoid acids (up to 90% purity) with liquified hydrocarbon, for instance using propane or butane.

The crystalline cannabinoid acids contained may be used directly from the extractor, decarboxylated to yield neutral cannabinoids, esterified, oxidized, reduced, isomerized, or otherwise transformed either chemically, thermally or photochemically into alternative cannabinoids\cannabinoid acids of interest. Because the method described herein does not require decarboxylation to achieve high extraction yields (particularly necessary for supercritical CO₂ extraction) and decarboxylation can occur with purified crystalline extracts (i.e. after extraction), the space utilization in a vacuum oven can be increased dramatically (ca. 5-fold compared to biomass containing 20% cannabinoid acid; 10-fold with 10% cannabinoid acid biomass) thus enhancing throughput. Decarboxylation of the highly pure extract also has the advantage of better heat transfer than decarboxylation within the biomass. Additionally, because the rate of decarboxylation can be greatly affected by the presence of other chemical species within the biomass (through matrix effects) and these species are not present in the extracted cannabinoid acids, shorter times and lower temperatures are permissible for decarboxylation (i.e. higher yields can be obtained). The purity of the crystalline material obtained from the extract is sufficiently high that no further processing is necessary to obtain a usable distillate with greater than 90% purity. Thus, precipitation of fats, waxes and other phytochemical impurities followed by distillation is not required resulting in a significant cost savings due to lower initial capital expense, shorter operations time and avoidance of massive losses in yield due to one or multiple distillations or recrystallizations to achieve the desired purity. Throughput can be roughly doubled by removing the time-consuming step of distillation. Furthermore, the purification with subcritical and/or supercritical CO₂ yields a mixture of terpenes and neutral cannabinoids that can be processed by standard means and sold.

In at least one embodiment, the impurities can be stripped out using other solvents (such as, for instance, fluorinated hydrocarbons like tetrafluoroethylene or slightly acidic/neutral water) that are selective to dissolution of terpenes and/or neutral cannabinoids so long as dissolution of cannabinoid acids is limited. Alternatively, solvents that are capable of solubilizing cannabinoid acids, but have a slower rate of solubilization relative to solubilization of impurities may be employed with special emphasis being placed on the time component of the stripping extraction. The parameters for subcritical and/or supercritical CO₂ stripping of impurities can be adjusted as needed to obtain either high yields with high purities of neutral cannabinoids and cannabinoid acids or under harsher conditions over longer periods of time to strip neutral cannabinoids like CBG, CBD, or THC from the biomass yielding only high purity THCA upon secondary extraction.

The cannabinoids obtained in the stripping process can be recovered through standard precipitation→distillation methods. Liquified hydrocarbon extraction of the stripped biomass yields an extract with composition consistent with the pre-extraction (stripping) parameters. For instance, a subcritical CO₂ stripping primarily removes terpenes from the biomass while a combination of subcritical and supercritical CO₂ stripping also removes neutral cannabinoids. The former resulting extract from these scenarios contains a mixture of neutral cannabinoids with total purities (THC+THCA) of up to 85% and the latter yielding an extract containing primarily cannabinoid acids with average purities of 90% for THCA (the remaining constituents being primarily CBGA and THC).

The PiggyBack extraction process is illustrated in FIG. 25.

Crude PiggyBack extract is solubilized in a polar solvent, preferably ethanol, in a mixing vessel. Shear mixing is required to ensure homogeneity as hard clumps of THCA crystals can form during the extraction process. Ethanol concentrations of at least 20% w/w in the final formulation were sufficient for cystal dissaloution and homogenization. and semi-automated packaging.

In accordance with the invention, the biomass obtained from the PiggyBack extraction process is first winterized. Winterization is the process of removing compounds, such as fats, lipids, waxes, and chlorophyll, from the crude oil before the distillation process. Winterization involves taking a nonpolar substance, i.e. crude oil, and dissolving it in a polar solvent, such as ethanol, at sub-zero temperatures to create a miscella mixture. Any polar solvent will work for this purpose, but ethanol is preferred. The ethanol should be added in a ratio of about 1:10 to 1:3 ethanol to biomass. In one embodiment, the ethanol is added in an amount such that the concentration does not exceed 5000 ppm, or 0.50 w/w %.

Once the ethanol is added to the biomass, the temperature of the mixture should be maintained at −20° C. or lower in a chiller or freezer for a time period of at least 24 hours to coagulate the undesirable ingredients. Once sufficiently coagulated, the miscella is filtered, preferably with the assistance of a vacuum, during which the ingredients should be kept cool to assure the lipids and waxes do not dissolve back into solution. Once filtered, the winterized PiggyBack extract (WPE) is collected and at least some of the ethanol is evaporated from the WPE.

In-house terpenes (IHT)(terpenes extracted from Cannabis or another plant/fruit/or other natural or synthetic source in the form of isolates or blends) are then added to the THCA mixture to contain up to 10% w/w IHT. The crystals are preferably mixed with at least 3% IHT, with 7-10% w/w IHT being preferred. It has been determined that, in terms of appearance, formulations containing 5, 7, and 10% w/w IHT were most acceptable, with about 10% IHT being preferred by patients in terms of taste. Formulations with at least 5% w/w IHT are preferred in terms of consistencies, i.e. the formulation being soft and crystalline enough to manipulate and transfer.

According to at least one embodiment, the slurry is then mixed with up to 25% ethanol. The slurry is preferably mixed with at least 15% ethanol, with 20% ethanol being preferred. According to at least one embodiment the mixture is then packaged, preferably using an apparatus such as the hopper and valve assembly shown in FIG. 1. The product is preferably packaged at a temperature of between about 10-40° C. and a pressure of between about 11-12 psi. In one embodiment, the product is packaged at a temperature of about 30° C. and a psi of about 11 psi. An actuating time of at least 1.75 seconds was sufficient for consistent dispense masses.

The mixture is then heated to a temperature of between about 40-80° C. (55-65° C. preferred) and placed under an incrementally increased vacuum for a time period of at least 8 hours for volatiles purge, and in certain embodiments preferably to a volatiles threshold of below about 0.36%. In one embodiment, a seed crystal is included in the mixture to facilitate recrystallization. The mixture is typically heated for at least an hour and up to 15 hours, and preferably with a vacuum, typically at psi of −1 to −14 psi (full vacuum), with about −10 to −14 psi being preferred. In one embodiment, the mixture is headed for about 12-18 hours. It has also been determined that the vacuum should not be pulled away too aggressively at the start of purging, but should ideally be pulled in 1 psi increments every 20-30 minutes starting at −10 psi to prevent rapid evaporation of the solvent from individual jars. The average post-purge mass typically ranges from about 3-10% RPD. As persons of ordinary skill in the art will understand, the term “purged” refers generally to the removal of a solvent via vacuum and/or heat.

According to at least one embodiment, the sugar wax product is then packaged, preferably using an apparatus such as the modified hopper and valve assembly shown in FIG. 1. The product is preferably packaged at a temperature of between about 40-60° C. and a pressure of between about 0.2-1.0 psi. According to certain embodiments, the product is packaged at a temperature of about 50° C. and a psi of about 0.5 psi and a starting actuating time of 0.90 seconds. Each packaged unit typically contains about 5 mg of THCA crystals added prior to recrystallization. The average post-purge mass typically ranges from about 3-10% RPD.

The following examples are offered to illustrate but not limit the invention. Thus, it is presented with the understanding that various formulation modifications as well as method of delivery modifications may be made and still are within the spirit of the invention.

### Example 1

### Preparation of Sugar Wax

### Materials and Methods

Biomass. All extract used in these experiments was obtained from biomass with 210730EOG-2 and 210916EOG batch ID's. Biomass was weighed at the time of harvest and hung to dry per GRO-002⁵. After four days of drying, biomass was destemmed and allowed to air dry until the moisture content was <10% (w/w) per PRO-001⁴ before further extraction and processing.

THCA crystals. All THCA crystals were obtained via PiggyBack extraction (sequential subcritical carbon dioxide (CO₂), then propane extraction) of dried, destemmed biomass per PRO-003³ and PRO-019², respectively.

Winterized PiggyBack extract (WPE). All WPE was obtained by winterizing THCA crystals with ethanol (EtOH) at a 1:5 mass ratio, filtering, and evaporating EtOH down to 50 mbar per PRO-004⁸. The amount of EtOH remaining in the WPE after evaporation was estimated to be 18 (±2) % (w/w), which will need to be removed during the vacuum purge objective.

Raw materials, equipment, and consumables. All raw materials, equipment and consumables needed to complete the methods are given in Tables 1-3.

TABLE 1
All raw materials necessary to complete the methods.

| Raw Material | Manufacturer/Supplier | Lot #/Batch ID |
|---|---|---|
| 9 mL jar closures | eBottles | 20210610 |
| 9 mL jars | eBottles | 1855 |
| Ethanol, USP grade, | Barton Solvents | 20A09/010920 |
| 190+ proof | | |
| In-house terpenes | MPI | IHT-01¹ |
| THCA crystals | MPI | 210916EOG-A, |
| | | 210825EOG-A |
| Winterized PiggyBack | MPI | 210730EOG-2-A-P |
| Extract (WPE) | | |

TABLE 2
All equipment necessary to complete the methods.

| Equipment | Manufacturer/Supplier | Model # |
|---|---|---|
| ¼″ MNPT × ⅛″ FNPT adapter | McMaster-Carr | 48805K263 |
| 18″ × 26″ baking sheet | Winco | ALXP-1826 |
| 18″ × 26″ baking sheet lid | Winco | CXP-1826 |
| ⅜″ male Swagelok × ¼″ | Swagelok | SS-600-7-4 |
| FNPT adapter | | |
| ⅜″ OD SS tubing | Grainger | 48KV06 |
| Air compressor | Husky | 3320445 |
| Analytical balance | Ohaus | AX224 |
| Chest freezer | Frigidaire | FFFC20M4TW |
| Circulating water bath | Thermo Scientific | TSCIR19 |
| Cold trap | Lab Society | LS-CTK-C |
| Filter flask set | Amazon | LB78-5/SET |
| Heat gun | Porter-Cable | PC1500HG |
| Homogenizer | Silverson | L5MA |
| Hot box | Benko Products | 6E1-CS |
| Immersion chiller | Polyscience | IP-35 |
| 3 L plastic pitcher | Fisher Scientific | 10-210-679 |
| 2000 mL evaporating flask | Fisher Scientific | CG151235 |
| Moisture balance | Ohaus | MB120 |
| Semi-Automated Dispenser | ATG Pharma | R-300L |
| One-way pneumatic | | |
| actuated valve | Swagelok | SS-4SKPS6-BN90-A15C343979 |
| Overhead mixer | Cole-Parmer | 5070230 |
| High Shear Homogenizer | Silverson | L5M-A |
| Mixing Blade | Fisher Scientific | |
| Portable oil vaporizer | Puffco | n/a |
| Precision balance | Ohaus | AX8201 |
| Ring stand | Fisher Scientific | 31-502-258 |
| Rotary evaporator, pilot scale | Buchi | R-205 |
| Rotary evaporator, lab scale | Heidolph | Hei-VAP |
| Silicone scraper | Amazon | n/a de |
| Two-prong extension clamp | Fisher Scientific | 05-769-6Q |
| Vacuum oven | Being | BOV-90 |
| Vacuum pump | Vacuubrand | PC3001 |
| Vacuum pump | Robinair | 15500 |
| Vape filler | ATG Pharma | RL-300 |
| Vortex mixer | Fisher Scientific | 02-215-418 |

TABLE 3
All consumables necessary to complete the methods.

| Consumable | Manufacturer/Supplier | Item/Model # |
|---|---|---|
| 10-100 μL pipette | Fisher Scientific | M100E |
| 10-100 μL pipette tips | Fisher Scientific | F148415G |
| 100-1000 μL pipette | Fisher Scientific | M1000E |
| 100-1000 μL pipette tips | Fisher Scientific | F148180G |
| 15 mL centrifuge tubes | Fisher Scientific | 12-565-268 |
| 50 mL centrifuge tubes | Fisher Scientific | 06-443-19 |
| Cleanroom wipes | Uline | S-21888 |
| Disposable transfer pipette | Fisher scientific | 13-711-9CM |
| Foil trays for moisture balance | Fisher Scientific | 08-732-110 |
| HPLC vial labels | Avery | 6467 |
| HPLC vials | Fisher Scientific | 03-377-298 |
| Metal spatula | Fisher Scientific | 31-501-960 |
| Methanol, HPLC grade | Fisher Scientific | A454-4 |
| Parafilm | Fisher Scientific | S37440 |
| Quarantine labels | Avery | |

Volatiles analysis on the moisture balance. Triplicate 1.00 g (±0.05 g) samples of THCA crystals were spiked with either 0, 2.5, 5, 10, 15, or 50 μL of EtOH and analyzed on the moisture balance. The actual volatiles content expressed as [EtOH] (ppm) was plotted vs the expected values to determine if the results were 1) linear and 2) in agreement with the expected values. This experiment was performed to determine if the moisture balance could be used to reliably quantify the amount of volatiles (i.e., EtOH) in a formulation before and after vacuum purging. The slope of the line for the actual vs expected [EtOH] (ppm) was used to set a threshold volatiles content never to exceed in a formulation such that it does not exceed 5000 ppm, or 0.50 w/w %, which is Iowa's residual EtOH specification for a vaporizable product.

Lab scale formulations. A bulk ˜30 g amount of WPE was analyzed in triplicate on the moisture balance to determine the percent volatiles (i.e., % volatiles) and % solids in the starting raw material. WPE samples (1.25 g (±0.10 g)) were aliquoted and mixed with in-house terpenes (IHT) such that the final formulations contained 0, 1, 3, 5, 7 and 10% (w/w) IHT. Each formulation was warmed up in the hot box at 60° C. for 30 min before vortexing for 30 sec, or until the formulation looked consistent throughout. The amount of IHT added was determined based on the mass of solids (g) in the formulation from the % solids calculation (Eq. 1). The formulations containing 0, 1, and 3% IHT were purged in the vacuum oven at 60° C. for 2-, 4-, and 8-h increments. The formulations containing 5, 7, and 10% IHT were purged in the vacuum oven at 60° C. for 8 h. The amount of volatiles in each formulation was determined on the moisture balance after purging to see if they were below the threshold (see section Volatiles analysis on the moisture balance). Each formulation was then tested by a panel of three cardholders to determine their level of acceptability in the following categories: appearance, taste, effect, and consistency. Taste was particularly important to characterize as residual EtOH is the number one concern with this formulation, and the product cannot taste like EtOH even if the volatile content is beneath the threshold. Cardholders were asked to rate their level of agreement with the following statement: “The [category] of the formulation was acceptable.” Acceptability scores were assigned according to the scale in Table 4. Each category must receive an average score of 4 or better to be deemed acceptable. The formulation with the highest score was chosen for pilot experimentation.

% Solids=100%−% Volatiles Equation 1.

### Calculating the Percent Solids in the Starting Amount of WPE for Formulation of a Batch of Sugar Wax

TABLE 4
Scale for ranking the acceptability of
each category during sensory testing.

| 1 | 2 | 3 | 4 | 5 |
|---|---|---|---|---|
| Strongly | Somewhat | Neither Agree | Somewhat | Strongly |
| Disagree | Disagree | Nor Disagree | Agree | Agree |

Pilot scale mixing. A 93 g sample of WPE evaporated down to 50 mbar on the rotovap was analyzed in triplicate on the moisture balance to determine the percentage volatiles in the raw material. The percent solids and mass of solids (g) in the formulation was determined from these measurements. IHT was then added to the WPE such that the final formulation was 90% solids and 10% IHT (w/w). The formulation was mixed on an overhead mixer at 300 rpm for 15 min and then placed in the vacuum oven at 50° C. and atmospheric pressure until it was ready to be packaged into individual jars.

Semi-automated dispensing on the vape filler. The final appearance of the packaging apparatus is shown in FIG. 1. The bowl reducer was set up to serve as the hopper on the vape filler (FIG. 1A). A 2″ piece of product tubing was cut and attached to the bottom portion of the bowl reducer (FIG. 1B). The one-way pneumatic valve was equipped with the necessary tubing and fittings to allow product to flow through the 2″ piece of product tubing from the bowl reducer through the valve (FIG. 1C). A small 1″ piece of product tubing was attached to the outlet of the one-way valve with a ¼″ NPT×⅛″ quick connect to direct the flow of formulation out of the valve and into a glass jar (FIG. 1D). The bowl reducer, product tubing, and one-way valve were all wrapped with electric heat tape set to 50° C. The formulation was then removed from the hot box and transferred into the bowl reducer. A heat gun was used on the lowest setting to facilitate this transfer. The lid was secured to the bowl reducer with a high-pressure clamp and the system was hooked up to the compressed air line with the regulator set at 0.5 psi. Blue tubing from the digital control box was connected to the one-way pneumatic valve and set to actuate at 0.50 sec.

The actuating time was adjusted until the dispensed mass was between 1.20-1.25 g for three consecutive measurements to calibrate the apparatus prior to packaging the pilot scale batch. Individual 9 mL jars were then filled using the purge button on the vape filler until the formulation ran out. An empty mass and final mass of each jar were taken to track the mass loss of each jar during purging in the vacuum oven. The formulation collected during calibration of the packaging apparatus was loaded back into the bowl reducer towards the end of the pilot batch to reduce product loss. Individual components of the packaging apparatus were also weighed before and after packaging to determine the amount of product loss we can expect in a full-scale production batch.

After packaging, four random jars were pulled and analyzed on the moisture balance to determine the baseline amount of volatiles in the formulation. This value was compared to the baseline volatiles obtained on the lab scale to check for formulation consistency during scale up. These jars were then tested in triplicate for potency per QCU-0106 and QCU-0267 to determine if the total THC before purging was consistent during scale up as well.

Amongst the remaining jars, 5-10 mg of THCA crystals from batch ID 210825EOG-A were added to ten jars to serve as a “seed” crystal prior to recrystallization. Both sets of jars (with and without a seed crystal) were placed in the freezer (−10° C.) for 14 h to facilitate the precipitation of THCA from the formulation. The goal of adding a seed crystal to a select number of jars was to determine if this would result in a more acceptable final product appearance and consistency after vacuum purging.

Vacuum purging on the pilot scale. Each jar was then loaded uncapped into the vacuum oven. The jars were spaced evenly throughout the oven and on all three shelves. The oven was set to 60° C. and vacuum was pulled in 1 psi increments every 20-30 min starting at −10 psi to −14 psi, or full vacuum. Vacuum was kept static throughout this objective. After 9 h and 15.5 h in the vacuum oven three sets of jars with and without a seed crystal were removed and analyzed for volatiles and potency. All jars with a seed crystal were removed from the vacuum oven after 15.5 h after determining that each jar was beneath the threshold of volatiles and had an acceptable appearance and consistency. The remaining jars without a seed crystal were purged for an additional 24 h (40 h total). After 40 h, the remaining 46 jars were then seeded with −5 mg of THCA crystals, placed in the freezer for an additional 14 h and purged for an additional 7 h at 60° C. and full vacuum (total purge time 47 h).

At the conclusion of the pilot experiment, 6×1 g jars with a seed crystal removed at 15.5 h and 6×1 g jars that were purged for 47 h were set aside for sensory testing per the template provided in Attachment 2. The jar-to-jar variation per patient per category were assessed to look for statistical differences in overall final product acceptability when a seed crystal was added prior to recrystallization. An additional 42×1 g jars were set aside for room temperature and refrigerated stability testing per QCU-017.

### Results

Volatiles analysis on the moisture balance. Measuring a known amount of EtOH that was spiked onto THCA crystals using the moisture balance showed linearity from 0-10 μL, or 0-˜10,000 ppm, of EtOH as shown in FIG. 2A. This method did not produce linearity beyond 10,000 ppm (FIG. 2B) for unknown reasons despite two triplicate attempts at 15 and 50 μL (˜13,000 and ˜39,000 ppm). Given the residual EtOH specification of 5000 ppm was within the linear range, it was determined that the moisture balance was an acceptable method for determining the amount of volatiles (i.e., EtOH) in a sample. Using the slope of the line generated from the actual [EtOH] (ppm) vs expected [EtOH] (ppm) along with the maximum percent error between the actual and expected values within this region (16%), we were able set a threshold of 0.36 w/w % (i.e., 3600 ppm) volatiles never to exceed in our formulation after purging in the vacuum oven.

Lab scale formulations. Vacuum purge conditions were initially defined using the lab scale formulations prepared with 0, 1, and 3% IHT (w/w). WPE exhibited a baseline volatiles of 2.0 (±1.2) % as shown in FIG. 3. A 60% error was less than ideal, but it was consistent with the non-linearity observed beyond 10,000 ppm on the moisture balance. Ideally, more measurements would have been taken at each time interval if it were not for the high cost of the raw material and minimum sample amount on the moisture balance (≥0.5 g). After two h in the vacuum oven, only the 3% formulation was above the threshold. After 4 h, the 0, 1, and 3% formulations all had a volatiles content <1% but were above the threshold. After 8 hours, only the 3% formulation was above the threshold. From this information, we were able to establish that a minimum of 8 hours would be necessary to purge enough EtOH from our formulation. A mass loss of 11 (±1) % was observed regardless of formulation ratio or vacuum purge duration on the lab scale.

The scores for the appearance of each formulation are shown in FIG. 4. The formulations containing 7% and 10% IHT scored higher than the formulations containing only 3% IHT for all three patients. The formulation with 10% IHT was chosen for piloting partially because it had more of a sugary, crystalline appearance, whereas the formulations prepped w/<5% IHT had more of a dull, waxy appearance (FIG. 5). The low score for the appearance of the second 3% IHT lab scale formulation (i.e., 3% (2)) was a result of pulling vacuum too quickly at the start of purging. It was discovered that vacuum should be pulled in 1 psi increments every 20-30 min starting at −10 psi to prevent rapid evaporation of EtOH from the individual jars.

The taste of each lab scale formulation is shown in FIG. 6. Once again, the formulations containing 5, 7, and 10% IHT were deemed most acceptable. Anecdotal evidence from the patients stated that they preferred the taste of the formulation containing 10% IHT the most.

The effect of each lab scale formulation was deemed acceptable regardless of formulation ratio (FIG. 7). No formulations could be ruled out based solely on effect.

The consistency of each lab scale formulation closely mirrored the appearance acceptability as shown in FIG. 8. Consistency showed a general improvement from 0-5% IHT, with exception to the second lab scale formulation prepped at 3% IHT. At 5% IHT, the consistency of the formulation was sufficient such that no further improvements were made with increasing terpene content. The consistencies of the formulations with 0, 1, and 3% IHT were too hard and brittle and difficult to transfer from the jar to the vaporization device. At 5+% IHT, the formulation was soft and crystalline enough to make manipulation and transfer of the final product easy to achieve.

Based on all sensory data and vacuum purge data gathered on the lab scale formulations, the final formulation ratio of 90% WPE/10% IHT and vacuum purge conditions of 8 hours at 60° C. under gradual static vacuum from −10 psi to −14 psi (i.e., full vacuum) were chosen as the conditions to pilot.

Pilot scale mixing and semi-automated dispensing. A 103 g formulation consisting of ˜90% WPE and 10% IHT (w/w) was successfully mixed and packaged on the apparatus shown in FIG. 1. The experiment produced 67 units at an average mass of 1.22 (+0.05) g, or a relative percent difference (i.e., RPD) of only 4%. We were targeting a mass of 1.20-1.25 g to account for the evaporation of EtOH and other volatiles during purging, which accounted for 11% of the pre-purge formulation mass on the lab scale. There were only two rejects during filling, and a mass loss of 19.0 g, which will remain constant as batch size increases during scale up. In all, the unit yield and mass yield of this experiment were both 95%, indicating use of the packaging apparatus resulted in only 5% loss.

The pre-purge volatiles content in the pilot batch and lab scale batches are shown in FIG. 9 for comparison. The amount of volatiles before purging during scale up was consistent at 6 (±2) %. Once again, there was a high degree of variation between samples on the lab and pilot scale with a 27% RPD for both sets of measurements.

The potencies (w/w %) of THC, THCA and total THC (Total THC %=THC %+THCA %*0.877) for the lab scale and pilot scale formulations prior to purging in the vacuum oven are shown in FIG. 10. There were no significant differences in cannabinoid potencies during scale up, however the RPD for THCA % and total THC % on the pilot scale were higher than desirable at 15% and 16%, respectively.

Vacuum purging on the pilot scale. The first round of pilot scale vacuum purging was unsuccessful and resulted in a final product that was inconsistent in both appearance and consistency as shown in FIG. 11. This was believed to be a result of 1) adding IHT to THCA crystals prior to winterizing as opposed to after and 2) a lack of recrystallization between packaging and purging in the vacuum oven. A second pilot batch was attempted where IHT was added to WPE after winterization as opposed to the THCA crystals before winterization. To promote recrystallization of this batch, 10/67 packaged units had a THCA seed crystal added to them after packaging. After 9 h in the vacuum oven, the jars with a seed crystal had a volatiles content beneath the threshold of 0.36% (FIG. 13). The appearance of these jars was too inconsistent to be deemed acceptable (FIG. 12), so they were left in the oven to purge longer. Some of the jars had achieved sufficient recrystallization (FIGS. 12D and 12E), while others required more time under vacuum (FIG. 12F). The jars without a seed crystal had neither an acceptable appearance (FIG. 12A-C) or were sufficiently purged of EtOH, as their average % volatiles exceeded the threshold.

After 15.5 hours of purging, vacuum was broken again to check the progress of the appearance and % volatiles of the jars with and without a seed crystal. The appearance of the jars without a seed crystal was still unacceptable, although it was noted that small amounts of precipitate had begun forming in some of the jars (FIG. 14A). Further purging improved the consistency of the appearance of the jars with a seed crystal (FIG. 14D-F), so these jars were removed from the vacuum oven. The amount of volatiles in each set of jars was beneath the threshold as shown in FIG. 15.

After 15.5 h of purging, all jars with a seed crystal were removed from the vacuum oven because their appearance and % volatiles were acceptable. The jars without a seed crystal were loaded back into the vacuum oven and purged for an additional 24 h for a total of 40 h of purging. After 9 h under vacuum, it was hypothesized that the absence of a seed crystal slowed the drying of these formulations (FIG. 13) and that more time in the oven would allow the formulations to slowly precipitate and transform their appearance. This theory was disproven at 15.5 h, when the unseeded jars measured lower % volatiles (FIG. 15) and still did not have an acceptable appearance (FIG. 14A-C). Although 40 h of drying did improve the appearance of the non-seeded units (FIG. 16), it was still not acceptable, indicating the addition of a seed crystal would be a necessary step during full scale production after packaging individual units on the vape filler. Triplicate volatiles measurements were taken on the non-seeded jars after 40 h of purging, and all three measurements came back at 0.00%, indicating the units were sufficiently purged of residual EtOH. One theory as to why the addition of a seed crystal was necessary only on the pilot scale was the choice of rotovap. WPE was taken from a production batch that was evaporated on a 20 L Heidolph rotovap, while each pilot scale batch was evaporated on a 2 L Buchi rotovap. The WPE used in the lab scale experiments was noted to already have THCA crystals precipitating out of the formulation before terpenes were added, whereas the WPE on the pilot scale did not. It is suspected that the size of the rotovap and the amount of winterized oil being evaporated played a role in the final EtOH content in the WPE, although this will have to be tested during the first full scale production batch. The following support this hypothesis: 1) the seeded jars in the pilot batch took longer to achieve an acceptable appearance than the lab scale batch and 2) the total mass loss after purging on the lab scale was 10.8 (+0.3) % vs. 15 (+1) % for the seeded jars in the pilot batch after 9 h in the vacuum oven. The ˜25% variation in total THC observed on the pilot scale (FIG. 10) likely also contributed to the lack of consistency in appearance of the pilot scale batch and why adding a seed crystal was necessary to facilitate recrystallization.

Seeding the remaining jars after 40 h of purging did not produce an identical appearance to what we saw with the seeded jars at 15.5 h of purging (FIG. 17). This is likely a result of the extent of decarboxylation that occurred due to prolonged exposure in the vacuum oven (FIG. 18). These jars were much oilier and stickier in appearance, both of which are qualities of THC, which made the jars' appearance and consistency less than acceptable.

Potency analysis of the sugar wax. THC %, THCA %, and total THC % for the lab scale and pilot scale formulations are shown in FIG. 18. A separate set of triplicate potency samples were analyzed for each vacuum purge duration and each type of sample (i.e., with or without a seed crystal). Also presented are the potency values from the lab scale experiment on the formulation with 10% IHT for comparison. The total THC % across all jars were statistically the same during the lab and pilot scale with the following exception: 9 h under vacuum led to statistically lower total THC % in the jars without a seed crystal (73±2%) than the jars with a seed crystal after 15.5 h under vacuum (79±1%). There was a statistically significant increase in THC % between the unseeded jars from 9 to 15.5 to 40 hrs in the vacuum oven. Although total THC remained the same in these sample sets, this much decarboxylation should be avoided due to the sticky nature of THC, which lowers the acceptability of the appearance and consistency of the final product. Decarboxylation was not the only factor affecting the appearance of the unseeded jars as an acceptable appearance was not observed at 9 h or 15.5 h when the THC % was statistically lower than at 40 h. It is unknown why the unseeded jars did not recrystallize at any point in this study. The lab scale formulation was unseeded but was noted to have precipitate (i.e., a “seed”) already forming after EtOH evaporation on the 20 L rotovap. Potency analysis in FIG. 10 shows that this formulation was more homogeneous than the pilot scale formulation with a total THC % variation of <10% vs 25%. Perhaps both the scale and equipment selection for EtOH evaporation as well as the mixing protocol between the lab and pilot scale worked in conjunction to produce the lack of recrystallization we saw in the unseeded jars on the pilot scale.

Sensory testing of the sugar wax. The acceptability of the appearance of the pilot scale formulation is shown in FIG. 19. Five of the six patients found the appearance of the jars with a seed crystal to be acceptable, whereas no patients found the appearance of the unseeded jars to be acceptable. We saw a statistical difference in the acceptability of the appearance between the seeded and unseeded jars for five out of the six patients.

The acceptability of the taste of the pilot scale formulation is shown in FIG. 20. Four out of the six patients found the taste of the seeded jars to be acceptable, while three out of the six found the taste of the unseeded jars to be acceptable. A statistical difference in taste between the seeded and unseeded jars was found in two of the six patients.

The acceptability of the effect of the pilot scale formulation is shown in FIG. 21. All six patients found the effect of the seeded jars to be acceptable, whereas five of the six patients found the effect of the unseeded jars to be acceptable. There was statistical a difference in the acceptability of the effect between seeded and unseeded jars for two of the six patients, with the seeded jars scoring higher both times.

The acceptability of the consistency of the pilot scale formulation is shown in FIG. 22. The largest discrepancy in results was in this category. There was a statistical difference between the acceptability of the consistency between the seeded and unseeded jars for three of the six patients, with the seeded jars scoring higher each time. The consistency of the seeded jars was deemed acceptable for five of the six patients, while the unseeded jars had a consistency that was acceptable for only two of the six patients.

Total acceptability of the pilot scale formulation is shown in FIG. 23. A minimum score of 16/20 was necessary for the formulation to be deemed acceptable. Statistical differences in the total acceptability between the seeded and unseeded jars were found with five of the six patients, with the seeded jars scoring higher. Five of the six patients found the seeded jars to be acceptable, whereas only two out of the six patients found the unseeded jars to be acceptable.

Stability testing of the sugar wax. Refrigerated and room temperature stability testing could not be performed on the sugar wax from the pilot scale batch because its duration in the vacuum oven decarboxylated more THCA to THC than what will be seen in a large-scale production batch. The appearance and consistency of the remaining jars was also different than our goal, so there would be no way to determine how these qualities change over time. Stability testing should proceed with the first large-scale production batch per the steps described in the Methods (see section Vacuum purging on the pilot scale).

### DISCUSSION

Of the 67 packaged pilot scale units prepared, the average post-surge mass was 1.0 (0.1) or a RPD of 10%, indicating the reproducibility of the post-purge masses regardless of vacuum purge duration or the presence of a seed crystal. In addition, evaluating just the jars packaged under the ideal conditions for a full production batch, the average post-purge mass was even more precise at 1.01 (±0.03) g, or a RPD of 3%. The total mass loss after vacuum purging at 15.5 h and 47 h was 18 (±1) % and 19.4 (±0.8) %, respectively, suggesting sufficient removal of EtOH from the starting material (18±2%). All masses from the pilot batch can be found in Attachment 3. The data collected on both the lab and pilot scale experiments indicate that the Sugar Wax formulation is ready for scale up to a full production batch (˜900 g) under the following conditions: 1) the formulation consists of 90% THCA crystals and 10% IHT (w/w), 2) the formulation gets packaged with the apparatus shown in FIG. 1 at 25° C., 11 psi, and a starting actuating time of 1.75 sec, which will be adjusted during priming and calibration of each batch, 3) each packaged unit gets ˜5 mg of THCA crystals added prior to recrystallization, and 4) each packaged unit gets vacuum purged for 15 h at 60° C. gradually pulling vacuum from −10 psi to −14 psi, or full vacuum, over the first two h. To test our hypothesis that EtOH evaporation was different on the lab and pilot scale due to rotovap selection, a small sample of jars (˜10) from the first production batch will not be seeded before recrystallization.

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplishes at least all of the intended objectives.

1. A process of manufacturing a Cannabis concentrate comprising the following steps:
obtaining an extract from Cannabis biomass using a closed-loop extraction process, said extraction process comprising sequential subcritical carbon dioxide with butane or propane;
mixing a composition containing one or more terpenes with the extract to form a mixture; and
heating the mixture to a temperature of between about 40 to 80° C. to form a Cannabis concentrate.

2. The process of claim 1 further comprising a winterizing step, wherein a winterized piggyback extract (WPE) is obtained by dissolving the Cannabis biomass in a polar solvent to form a dissolved biomass, cooling the dissolved biomass to form coagulated compounds and removing the coagulated compounds from the dissolved biomass to form the WPE.

3. The process of claim 2 wherein the polar solvent is ethanol.

4. The process of claim 3 wherein the Cannabis biomass is dissolved in a concentration of ethanol not exceeding about 0.50 w/w %.

5. The process of claim 1 wherein the terpenes comprise terpenes extracted from Cannabis or another plant, fruit, or other natural or synthetic source in the form of isolates or blends.

6. The process of claim 1 wherein the terpenes are mixed with the extract in an amount of up to about 20% w/w.

7. The process of claim 2 wherein the WPE is mixed with about 7 to 10% w/w terpenes.

8. The process of claim 1 wherein a seed crystal is mixed with the extract and the terpenes.

9. The process of claim 1 wherein the extract and the terpenes are purged for a time period of at least 8 hours.

10. The process of claim 7 wherein the WPE and the terpenes are purged for a time period of between about 12 to 18 hours.

11. The process of claim 1 wherein the mixture is heated under a vacuum.

12. The process of claim 11 wherein the vacuum is applied at a psi of about −1 to −14 psi.

13. The process of claim 1 further including the step of packaging the Cannabis concentrate at a temperature of between about 20 to 50° C. and a pressure of between about 8 to 12 psi.

14. The process of claim 1 wherein the Cannabis concentrate comprises about 5 to 10 mg of THCA crystals.

15. The process of claim 1 wherein resulting average post-purge mass ranges from about 3 to 10% RPD.

16. The process of claim 1 wherein THCA seed crystal is added to the Cannabis concentrate after packaging.

17. A Cannabis concentrate manufactured using the process of claim 1.

18. The Cannabis concentrate of claim 17 comprising no more than about 0.36 w/w % volatiles.
